(19) United States
(12) Patent Application Publication
(10) Pub. No.: US 2017/0334806 A1
(43) Pub. Date: Nov. 23, 2017

(54) PRODUCTION OF BASESTOCKS FROM PARAFFINIC HYDROCARBONS

(71) Applicant: Emerging Fuels Technology, Inc., Tulsa, OK (US)

(72) Inventor: Kenneth L. Agee, Tulsa, OK (US)

(73) Assignee: Emerging Fuels Technology, Inc., Tulsa, OK (US)

(21) Appl. No.: 15/602,308

(22) Filed: May 23, 2017

Related U.S. Application Data
(60) Provisional application No. 62/340,241, filed on May 23, 2016.

(51) Int. Cl. C07C 2/12; C07C 5/03; C07C 7/00; C10M 105/04; C07C 45/50; C10L 1/08; C07C 1/22; C07C 4/04; C07C 7/04

(52) U.S. Cl. CPC C07C 2/12; C07C 4/04; C07C 5/03; C07C 7/00; C07C 7/04; C07C 45/505; C10L 1/08; C07C 1/22; C10M 105/04; C07C 2527/1213; C07C 2529/06; C07C 2529/40; C10L 2200/0484; C10L 2270/026; C10L 2290/543; C10M 2203/022; C10M 2203/1006; C10M 2203/024

(57) ABSTRACT

A process to convert paraffinic feedstocks into renewable poly-alpha-olefins (PAO) basestocks. In a preferred embodiment of the invention, renewable feed comprising triglycerides and/or free fatty acids are hydrotreated producing an intermediate paraffin feedstock. This paraffin feedstock is thermally cracked into a mixture of olefins and paraffins comprising linear alpha olefins. The olefins are separated and the un-reacted paraffins are recycled to the thermal cracker. Light olefins preferably (C2-C6) are oligomerized with a surface deactivated zeolite producing a mixture of slightly branched oligomers comprising internal olefins. The heavier olefins (C6-C16) are oligomerized, preferably with a BF3 catalyst and co-catalyst to produce PAO products. The oligomerized products can be hydrotreated and distilled together or separate to produce finished products that include naphtha, distillate, solvents, and PAO lube basestocks.

### CROSS-REFERENCE TO RELATED APPLICATIONS

This application claims priority to and the benefit of U.S. Provisional Patent Application Ser. No. 62/340,241, filed May 23, 2016, which is herein incorporated in its entirety by reference.

### BACKGROUND OF THE INVENTION

### 1. Field of the Invention

The present invention relates to a process and system to produce Group III and Group IV basestocks from paraffinic hydrocarbons. Such paraffinic hydrocarbons can be produced, for example, by a Fischer Tropsch process or can be produced by hydroprocessing a renewable feedstock, such as a triglyceride, a free fatty acid or mixtures thereof. Hydroprocessing may include hydrodeoxygenation, decarboxylation, and saturation, and may also be referred to herein as hydrotreating.

### 2. Description of the Related Art.

Fischer Tropsch syncrude, preferably syncrude made by a non-shifting Fischer Tropsch catalyst, comprises predominately normal paraffins (n-paraffins) also referred to as straight chain hydrocarbons. While the Fischer Tropsch synthesis, preferably with a non-shifting Fischer Tropsch catalyst, produces a broad range of carbon distribution from C1 to approximately C100, it produces, however, a small range of variation in molecular structure. Such molecules are predominately straight chain paraffins with lesser amounts of alpha olefins and primary alcohols. The alpha olefins and primary alcohols can easily be saturated yielding a range of high purity n-paraffins. Naturally occurring triglycerides, including fatty acids, can be hydroprocessed to produce predominately paraffin hydrocarbons of narrow boiling range.

The prior art teaches that these high purity n-paraffin molecules can be refined into paraffin solvents, oils, and waxes. The waxes can also be hydroisomerized into high quality basestocks. Such high quality basestocks are referred to in the market as Group III basestocks. The Group III designation is a formal industry term. The American Petroleum Institute designates lubricant base oils as follows: Group I, Group II, Group III and Group IV. As the quality of the basestock that can be produced from a high purity Fischer Tropsch wax often exceeds the quality of a typical Group III basestock, the basestock produced by hydroisomerization of Fischer Tropsch waxes may be known in the market by the informal term Group III+. These basestocks are highly desirable products and therefore represent one of the highest value products that can be produced by a Fischer Tropsch process.

As known to those skilled in the art, the hydroisomerization of Fischer Tropsch waxes to produce Group III or Group III+ basestocks will result in cracking a portion of the wax to lighter (lower molecular weight, lower carbon number) iso-paraffinic products, too light to be included in the Group III basestock products. Such light products may be further processed and finished as solvents, distillates or drilling fluids.

Another desirable group of products in the market are known as Group IV basestocks. Group IV basestocks are made by oligomerization of linear alpha olefins. These linear alpha olefins are commercially produced by oligomerization of ethylene to higher olefins. Most commercial Group IV basestocks (also known as polyalphaolefins or PAO) are made by oligomerization of 1-decene, which is a small fraction of the products of ethylene oligomerization.

Historically, alpha olefins have also been made by thermally cracking petroleum waxes. Such thermal cracking of petroleum waxes will yield a distribution of alpha olefins with a substantial portion in the C6 to C16 range. In U.S. Pat. Nos. 5,136,118 and 5,146,022, a process is demonstrated whereby petroleum waxes are thermally cracked into alpha olefins. The C6 to C16 olefins are further oligomerized into Group IV basestocks with properties similar to basestocks made from 1-decene. Some prior art processes, such as U.S. Pat. No. 8,440,872, have proposed a process that will convert a narrow fraction of a Fischer Tropsch syncrude into Group IV basestocks.

It is an objective of the present invention to convert a broad range of paraffin feedstocks into Group III and Group IV basestocks. When the paraffin source is from a Fischer Tropsch reaction, it is an objective to provide a process that will convert a major portion of a Fischer Tropsch syncrude into Group III and Group IV basestocks.

### SUMMARY OF THE INVENTION

The present invention is a process designed to produce high yields of Group III and Group IV basestocks from paraffinic hydrocarbons, such as a Fischer Tropsch syncrude product, and/or from renewable feedstocks comprising triglycerides, diglycerides, monoglycerides, and free fatty acids. Heavy waxy Fischer Tropsch components are hydroisomerized, hydrotreated and distilled into one or more Group III basestock cuts. Lighter Fischer Tropsch molecules and/or certain renewable feedstocks are saturated to n-paraffins and then thermally cracked to produce a mixture of alpha olefins. Optionally, the very heavy C50+ waxy Fischer Tropsch components are not hydroisomerized but are also cracked to produce a mixture of olefins preferably in the C6 to C16 range. The appropriate range of olefins produced by cracking are oligomerized, hydrogenated, and fractionated to produce Group IV basestocks. Light olefins are dimerized, trimerized and/or oligomerized, including oligomerization over a surface deactivated catalyst to increase the average carbon number of the olefins. A portion of the light thermally cracked olefins and/or the higher olefins from the surface deactivated zeolite may be subjected to hydroformylation to alcohols followed by dehydration of the resulting alcohols to produce higher alpha olefins. Prior to dehydration, the hydroformylated product may also be subjected to mild hydrotreating to convert aldehydes to alcohols if necessary. A portion of the olefins produced by the surface deactivated zeolite will be internal olefins. The hydroformylation reaction with the appropriate catalyst will yield primary alcohols which upon dehydration will result in alpha olefins of one carbon number more than the starting olefins.

Therefore, the process makes it possible to convert light olefins (C2-C6) from thermal cracking into higher alpha olefins with an average carbon number of approximately 10 which are suitable for oligomerization to Group IV basestocks. Olefins can also optionally be used to alkylate an imported aromatic feedstock to make a polar aprotic blendstock useful for blending with Group III and Group IV basestocks of the present invention. Additionally, a minor portion of the olefins produced by thermal cracking may optionally be used to make a viscosity index (VI) improver.

### BRIEF DESCRIPTION OF THE DRAWINGS

FIG. 1 is a simplified process flow diagram showing the major components of a process and system according to a first preferred embodiment of the present invention.

FIG. 2 is a simplified process flow/diagram of a second preferred embodiment of the present invention.

FIG. 3 is a simplified process flow diagram of a third preferred embodiment of the present invention.

### DETAILED DESCRIPTION OF THE INVENTION

The embodiments discussed herein are merely illustrative of specific manners in which to make and use the invention and are not to be interpreted as limiting the scope of the invention. It is an objective of the present invention to make both Group III and Group IV basestocks in high yield from paraffinic feedstocks including a Fischer Tropsch syncrude and/or a renewable feed containing triglycerides or fatty acids. Group III basestocks of the present invention are made by hydroisomerization of heavy waxy paraffins, such as Fischer Tropsch wax components. Fischer Tropsch wax may be defined as the C20+ fraction of the Fischer Tropsch syncrude product. In the present invention, all or a portion of the Fischer Tropsch wax may be used to produce Group III basestocks by hydroisomerization. Other fractions of the Fischer Tropsch syncrude and/or the renewable feed may be thermally cracked to make alpha olefins that can be converted to Group IV and other basestock products. If it is desirable to produce more Group IV basestocks, some or all of the Fischer Tropsch wax may be separated by distillation, fractional crystallization, segmentation or any other separation process known to one skilled in the art and used as feed to one or more thermal crackers to produce additional alpha olefins which can be oligomerized to Group IV basestocks.

Waxy components not used for hydroisomerization and all or a part of the remaining lighter Fischer Tropsch fractions and/or any renewable materials, including triglycerides and fatty acids (after mild hydroprocessing), are used as feed to one or more thermal crackers to make olefins for oligomerization to Group IV and other basestocks. The resulting paraffinic intermediate products (either Fischer Tropsch derived or renewable) can be thermally cracked with good selectivity to linear alpha olefins. The resulting olefins will range from C2 to C20 or higher. Most commercial oligomerization processes designed to make Group IV basestocks start with 1-decene or a mixture of olefins with a narrow distribution centered around 1-decene. The process of the present invention uses one or more thermal crackers to make a range of predominately linear alpha olefins. The range of alpha olefins that is used for oligomerization to Group IV basestocks can be tailored to meet the requirements of the finished products. As such, olefins that are too heavy can be recycled to be hydrotreated and further cracked. Olefins that are of too low a carbon number are optionally modified by the process via a combination of oligomerization, hydroformylation and dehydration and, optionally, trimerization. The result is that light olefins of, for example, C2 to C6 can be converted to the appropriate range of alpha olefins, for example C7 to C17 for oligomerization to Group IV basestocks. The oligomers of the desired carbon range will be hydrogenated and, optionally, distilled to form synthetic iso-paraffinic lube range Group IV basestocks.

In a very limited embodiment ethanol, though not a paraffin, can be dehydrated to ethylene and converted to Group IV basestocks. The ethylene can be oligomerized to C4-C30 alpha olefins or trimerized to 1-hexene or both. Alpha olefins outside the target range of [C7-C17], for example, can be modified as follows:

C2—can be recycled and oligomerized or trimerized.

C3 to C5—can be oligomerized over a surface deactivated zeolite such as ZSM 5 or ZSM 23 and hydroformylated to the primary alcohol. The alcohol can be dehydrated to the 1-olefin.

C6—Can be hydroformylated and dehydrated to C7 alpha olefin.

C8-C16—Can be used as feed to the primary oligomerization reactor to make Group IV basestocks.

C18+—Can be saturated and used as feed to a thermal cracker to make more alpha olefins in the target range.

In a simplified embodiment of the present invention, the light olefins (C2-C6) can be oligomerized over a surface deactivated zeolite resulting in a product that can be hydrotreated and distilled into naphtha, high cetane distillate and lubricant or lube cuts. The lube fraction can be blended with PAO produced by oligomerization of the alpha olefins in the desired (C7-C17) range. This range can be adjusted to make the desired product.

In a preferred embodiment, when waxy components are hydroprocessed to make Group III basestocks, the lighter iso-paraffinic byproducts from hydroisomerization are separated from feed to the thermal crackers so that the alpha olefins produced for oligomerization to Group IV basestocks are highly linear, thus improving the quality of the Group IV basestocks.

In a preferred embodiment, when using Fischer Tropsch feed material, at least two and preferably three or more thermal crackers are used to crack the paraffinic Fischer Tropsch syncrude products due to the broad carbon distribution. Such Fischer Tropsch products may optionally be hydrotreated to saturate olefins and/or alcohols resulting in a highly paraffinic feed to the thermal crackers. The Fischer Tropsch products may also be separated or distilled into cuts, such as C5-C9, C10-C15 and C16-C20. Such separation makes it possible to better control the operating conditions of the thermal crackers to optimize yield to olefin products, preferably linear alpha olefin products. These thermal crackers may be operated on a once through basis or may be operated at lower conversion with separation and recycle of the unreacted paraffinic products. Such recycle operation makes it possible to optimize the yield of higher olefin products which will enhance the quality and yield of the Group IV basestock products.

Renewable paraffin feeds resulting from the hydroprocessing of triglycerides and fatty acids generally have a narrow carbon distribution making it easier to control thermal cracking in a single unit.

Thermal cracking of paraffin products from Fischer Tropsch syncrude and renewable feeds, such as hydroprocessed triglycerides, results in production of light olefins in the C2 to C6 range. These light olefins fall outside the target range of approximately C7 to C17 and well outside the more desired range of C8 to C14. The range C7 to C17 is used only as an example; within the process of the present invention this range can be adjusted according to the specific requirements to make high quality feedstock for oligomerization to Group IV basestock products over traditional catalysts such as BF3 or A1C13.

Therefore, it is an objective of the present invention to upgrade the light C2 to C6 olefins into the C7 to C17 range for feed to the oligomerization reactor. Upgrading of light olefins may include dimerization, trimerization and/or oligomerization over the appropriate catalyst. Oligomerization of light olefins may include reaction over a surface deactivated zeolite catalyst resulting in slightly branched olefins, a portion of which are in the desired lube oil range which, after hydrogenation, are useful as Group IV basestock products. Oligomerized internal olefins that are too light to be used as Group IV basestocks may be converted to alpha olefins by hydroformylation to primary alcohols followed by dehydration to alpha olefins, or may be used as feed to make an alkylated aromatic which can be used as a lubricant blendstock or, optionally, sulfonated and neutralized for use as a detergent.

Light olefins may be upgraded to alpha olefins of the desired carbon number (C7-C17) by one or more reactions. For example, ethylene may be trimerized to 1-hexene (C6 olefin) or oligomerized to C4+ alpha olefins. The C3-05 or C3-C6 olefins may be oligomerized over a small pore surface deactivated zeolite, such as ZSM5, ZSM-11, ZSM-23, or ZSM-48, to produce a range of slightly branched internal olefins. The C18+ fraction from the surface deactivated zeolite can be added to the finished Group IV product before hydrotreating and distillation. The C6 to C16 fraction contains some internal olefins. This fraction may be subjected to hydroformylation to produce primary alcohols over a Cobalt catalyst, followed by dehydration resulting in slightly branched alpha olefins of one higher carbon number. After hydroformylation, it may be necessary to hydrotreat trace aldehydes, converting them to alcohols before dehydration. The C5 and C6 olefins from thermal cracking may be added to the feed to the hydroformylation and dehydration reactors, resulting in a high yield of C6 and C7 olefins. Thus, light alpha olefins from C2 to C6 can be converted into alpha olefins in the C7 to C17 target range. The target range can be adjusted by control of distillation limits and recycle.

In a simplified embodiment, light olefins (C2-C6) may be oligomerized over a surface deactivated zeolite, resulting in slightly branched internal olefins. These internal olefins may be hydrotreated and distilled into naphtha, distillate and lube basestock cuts.

The oligomerization reaction of the present invention for the higher olefin feed, may be carried out in a batch or continuous process in a fixed bed or stirred tank reactor or any other type of reactor known to one skilled in the art. Any oligomerization catalyst known to one skilled in the art may be used, including catalysts comprising BF3, AlCl3, Ziegler, Cr/SiO2, metallocene and the like.

Products of the higher olefins oligomerization reactor may be finished by hydrotreating and distillation. Such products may be blended together in any portion to make basestocks for a variety of applications. Optionally, products may be separated. For example, products from a renewable feed may be processed separately if desired. In a preferred embodiment, the process will produce both Group III and Group IV basestocks of predominantly low viscosities in the 2 to 10 cSt (at 100C) range. The process will optionally also produce a smaller portion of higher viscosity, high VI, Group IV product (that can be used as a viscosity index VI improver) and/or an alkylated aromatic which can be used as a polar aprotic basestock or blending component.

The present invention makes it possible to convert paraffins, for example, from natural gas, coal or any carbonaceous feed via syn gas and Fischer Tropsch and/or from renewable feedstocks such as triglycerides and fatty acids into a mixture of products which can be blended to formulate a range of high quality synthetic lubricant products.

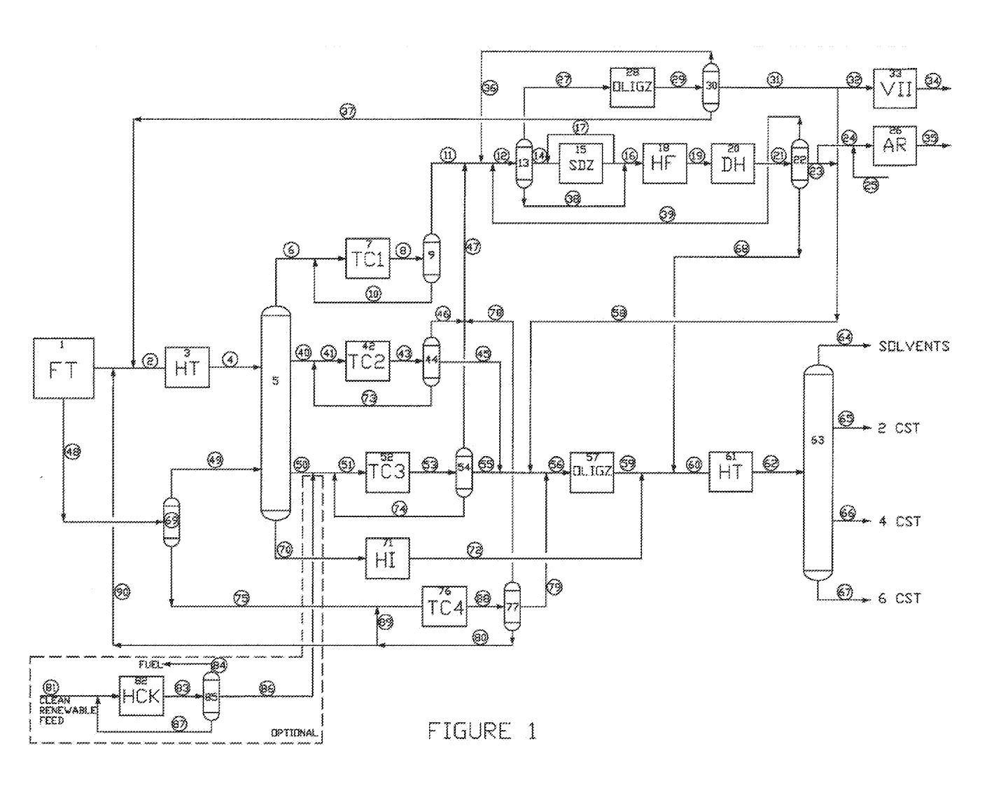

The process can be described by referring to FIG. 1, which is a process flow diagram representing a preferred embodiment. A Fischer Tropsch reactor (1) can be any type of reactor known to one skilled in the art, such as fixed bed, fluidized bed, micro channel or slurry bubble column. The preferred catalyst is a non-shifting catalyst with a high alpha preferably higher than 0.9, more preferably higher than 0.92. Carbon numbers given in brackets [ ] are for discussion purpose and not meant to be limiting.

Unprocessed product is removed from the Fischer Tropsch reactor system (1) in two streams. First light Fischer Tropsch (FT) syncrude [C5-C20] is transferred to hydrotreater (3) via line (2). Hydrotreater (3) saturates FT olefins and alcohols resulting in a very linear paraffinic product stream (4). Stream (4) is fed to a distillation column (5). Second heavy FT syncrude (48) [C20-C100] is fed to vacuum distillation column (69). A lighter fraction [C20-C49] is removed overhead in column (69) and transferred to distillation column (5) via line (49). Heavy waxy components [C50-C100] are removed from the bottom of distillation column (69) and transferred via line (75) to a thermal cracker 4 (76). Cracked product from thermal cracker 4 is transferred via line (88) to a separator (77). Separators, such as (77), may alternately be distillation columns or strippers. Light olefins [C6-] are removed overhead from separator (77). Higher olefins in the desired range [C7-C17] are removed as a side draw and transferred to the main oligomerization reactor feed line via line (79). Heavy cracked product (80) [C18+] may be recycled to thermal cracker 4 (76) via line (89) or recycled to feed hydrotreater (3) via line (90).

Light paraffinic hydrocarbons [C5-C9] are removed overhead from column (5) via line (6) and transferred to thermal cracker 1 (7). Cracked products are transferred via line (8) to column (9). The heavier mostly non-cracked [C7-C9] product is recycled from column (9) via line (10) to extinction. Light olefins mostly [C6-] are removed overhead from column (9) and transferred via line (11) to the light olefin processing section. Thermal cracker 2 (42) fed by stream (40) [C10-C15] and thermal cracker 3 (52) fed by stream (50) [C16-C20] operate like thermal cracker 1 (7) with one exception that columns (44) and (54) have side draws (45) and (55) respectively which transfer olefins of the desired range [C7-C17] to the main oligomerization feed line and therefore a detailed description is not necessary.

### Upgrading Light Olefins

Light olefins [C2-C6] may be upgraded to higher olefins [C7-C17] by several carbon-carbon bond forming steps described herein. Light olefins [C6-] are collected from each of the thermal crackers and transferred to column (13) via line (12). Ethylene is removed overhead from column (13) and transferred via line (27) to an oligomerization reactor (28). Ethylene can be effectively oligomerized to linear alpha olefins in the [C4-C30] range. Optionally, the ethylene can be reacted over a trimerization catalyst producing 1-hexene in high yield. The alpha olefin product produced by oligomerization reactor (28) is transferred via line (29) to column (30). Light olefins below the target set for higher olefins [C6-], for example, are removed overhead and recycled to column (13) via line (36). Light olefins, including [C3-C4] olefins removed via line (14), are subjected to oligomerization in a reactor (15) over a surface deactivated zeolite such as ZSM 5, ZSM-11, ZSM-23, or ZSM-48. The resulting product includes slightly branched internal olefins in the desired higher olefin range and a fraction of basestock range olefins [C18+]. Any unreacted monomer can be recycled via line (17).

The [C5-C6] olefins and [C6-] olefins being recycled to the process via line (39) are removed from the bottom of column (13) by line (38) and blended with the output of reactor (15) and then transferred via line (16) to a hydroformylation reactor (18) where olefins, including internal olefins, are converted to primary alcohols of one higher carbon number. Primary alcohols produced in reactor (18) are transferred via line (19) to a dehydration unit (20) where the alcohols are dehydrated to alpha olefins. Optionally, stream in line (19) can be subjected to mild hydrotreating (not shown on FIG. 1) to convert any aldehydes produced in reactor (18) into alcohols prior to dehydration (20). Olefin product from dehydration unit (20) is transferred via line (21) to a column (22) where olefins that are below the target range are removed overhead and recycled via line (39) back to column (13). Product that is above the target carbon range [C18+] is removed from the bottom of column (22) and transferred via line (68) to a hydrotreater (61). Olefin product in the target range is removed from column (22) via line (23) where it is combined with olefin product from ethylene oligomerization in the target range in line (31) and transferred to the inlet of oligomerization reactor (57) via lines (58) and (56). Optional Blendstock Production

Optionally, some of the alpha olefin product of line (31) can be transferred via line (32) to oligomerization reactor (33) to make a high VI viscosity index product (34). Oligomerization reactor (33) can use any oligomerization catalyst known to one skilled in the art, but preferably uses a chromium on silica catalyst. Another option of the process is to produce an aprotic blendstock using slightly branched olefins (23) which are transferred via line (24) to alkylation reactor (26). Aromatic feed is imported via line (25). The alkylated aromatic product (35) can be blended with the lube basestocks of the present invention. Production of Group IV Basestocks

Alpha olefins from thermal cracking that are in the target carbon number range [C7-C17], for example, are transferred to oligomerization reactor (57) feed line via lines (45), (55) and (79) where they are mixed with alpha olefins from light olefin upgrading (58). The mixed alpha olefin feed is transferred to oligomerization reactor (57) via line (56). Reactor (57) can use any catalyst known to one skilled in the art, but preferably uses a catalyst comprising boron trifluoride BF3. The resulting oligomers (59) are mixed with the [C18+] product (68) from column (22) and transferred to hydrotreater (61) via line (60). Hydrotreated product is transferred to column (63) via line (62) where it is separated into solvents which are removed via line (64) and various cuts of synthetic lubricant basestocks of different viscosities, such as 2 cSt removed via line (65), 4 cSt removed via line (66) and 6 cSt (67) (at 100C). These cuts may be varied to meet market requirements.

### Production of Group III Basestocks

Heavy waxy product exits the bottom of column (5) and is transferred to hydroisomerization reactor (71) via line (70). Hydroisomerization reactor (71) may include one or two stages or any configuration known to one skilled in the art and may use any hydroisomerization catalyst known to one skilled in the art. Hydroisomerized product is transferred to hydrotreater (61) via line (72) where it may be co-processed with Group IV basestock in line (60) or it may be campaigned through the hydrotreater (61) and distillation (63), producing a range of solvents and basestock products of different viscosities similar to the Group IV basestock products.

### Optional Renewable Feedstock

Optionally, a clean degummed feedstock comprising materials selected form the group comprising triglycerides, diglycerides, monoglycerides, and/or free fatty acids (81) is fed to hydrocracker (82) where it is converted in high yield to linear paraffins predominately [C10-C22]. Paraffin product is transferred to column (85) via line (83) where light products, including light hydrocarbons, water and carbon dioxide, are removed overhead. Heavy un-cracked product is removed from the bottom of column (85) and recycled to hydrocracker (82) via line (87). Paraffin product, predominately [C16-C22], are transferred to thermal cracker 3 (52) where it is processed with a paraffin fraction from column (5) of similar carbon distribution. Likewise, paraffin product in the [C10-C16] range is transferred to thermal cracker 2 (42) where it is processed with a paraffin fraction from column (5) of similar carbon distribution. The system as described will result in this portion of the finished Group IV basestocks produced being renewable.

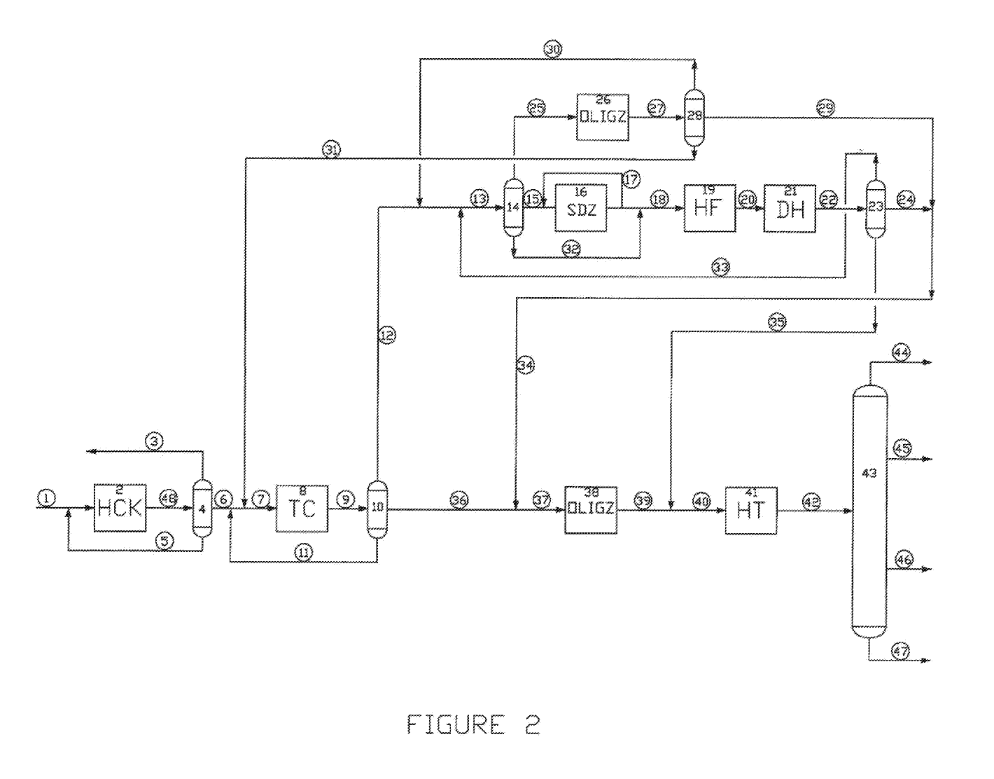

FIG. 2 represents another, second preferred embodiment of the present invention. Clean degummed renewable feedstock selected from the group comprising triglycerides, diglycerides, monoglycerides, and free fatty acids (1) is fed to a hydroprocessing unit (2). Hydroprocessed product is transferred via a line (48) to a column (4) where light products, including light hydrocarbons, water and carbon dioxide, are removed overhead. Heavy un-cracked product is removed from the bottom of column (4) and recycled to hydroprocessing unit (2) via line (5). Paraffin products, predominately [C10-C22], are transferred to thermal cracker 1 (8) via lines (6) and (7). Cracked product from the thermal cracker (8) is transferred via line (9) to a separator (10). Separator (10) may alternately be a distillation column or stripper. Light olefins [C6-] are removed overhead from (10) for further processing. Higher olefins in the desired range [C7-C17] are removed as a side draw and transferred to the main oligomerization reactor via a feed line (36). Heavy cracked product [C18+] may be recycled to thermal cracker 1 (8) via line (11).

Light olefins [C2-C6] are upgraded to higher olefins [C7-C17] by several steps described herein. Light olefins are collected from the thermal cracker and transferred to column (14) via line (12). Ethylene is removed overhead from column (14) and transferred via line (25) to oligomerization reactor (26). Ethylene can be effectively oligomerized to linear alpha olefins in the [C4-C30] range. Optionally, the ethylene can be reacted over a trimerization catalyst producing 1-hexene in high yield. The alpha olefin product produced by oligomerization reactor (26) is transferred via line (27) to a column (28). Light olefins below the target set for higher olefins [C2-C6], for example, are removed from the column (28) overhead and recycled to column (14) via line (30). Light olefins, including [C3-C4] olefins, are subjected to oligomerization (16) over a surface deactivated zeolite, such as ZSM5 ZSM-11, ZSM-23 or ZSM-48. The resulting product includes slightly branched internal olefins in the desired higher olefin range and a fraction of basestock range olefins [C18+]. Any unreacted monomer can be recycled via line (17). The [C5-C6] olefins (32) removed from the bottom of column (14) via line (32) are blended with the output of reactor (16) and transferred via line (18) to a hydroformylation reactor (19) where olefins, including internal olefins, are converted to primary alcohols of one higher carbon number. Primary alcohols produced in reactor (19) are transferred via line (20) to a dehydration unit (21) where the alcohols are dehydrated to alpha olefins. Optionally, stream (20) can be subjected to mild hydrotreating to convert any aldehydes produced in reactor (19) into alcohols. Olefin product from dehydration unit (21) is transferred via line (22) to column (23) where olefins that are below the target range are removed overhead and recycled via line (33) back to column (14). Product that is above the target carbon range [C18+] is removed from the bottom of column (23) and transferred via lines (35) and (40) to hydrotreater (41). Olefin product in the target range is removed from column (23) via line (24) where it is combined with olefin product from ethylene oligomerization in the target range via line (29) and transferred to the inlet of oligomerization reactor (38) via lines (34) and (37). Alpha olefins from thermal cracking that are in the target carbon number range [C7-C17], for example, are transferred to oligomerization reactor (38) via line (36) where they are mixed with alpha olefins from light olefin upgrading (34). The mixed alpha olefin feed is transferred to oligomerization reactor (38) via line (37). Reactor (38) can use any catalyst known to one skilled in the art but preferably uses a catalyst comprising BF3. The resulting oligomers removed via line (39) are mixed with the [C18+] product (35) from column (23) and transferred to hydrotreater (41) via line (40). Hydrotreated product is transferred to column (43) via line (42) where it is separated into solvents (44) and various cuts of different viscosities, such as 2 cSt (45), 4 cSt (46) and 6 cSt (47). These cuts may be varied to meet market requirements.

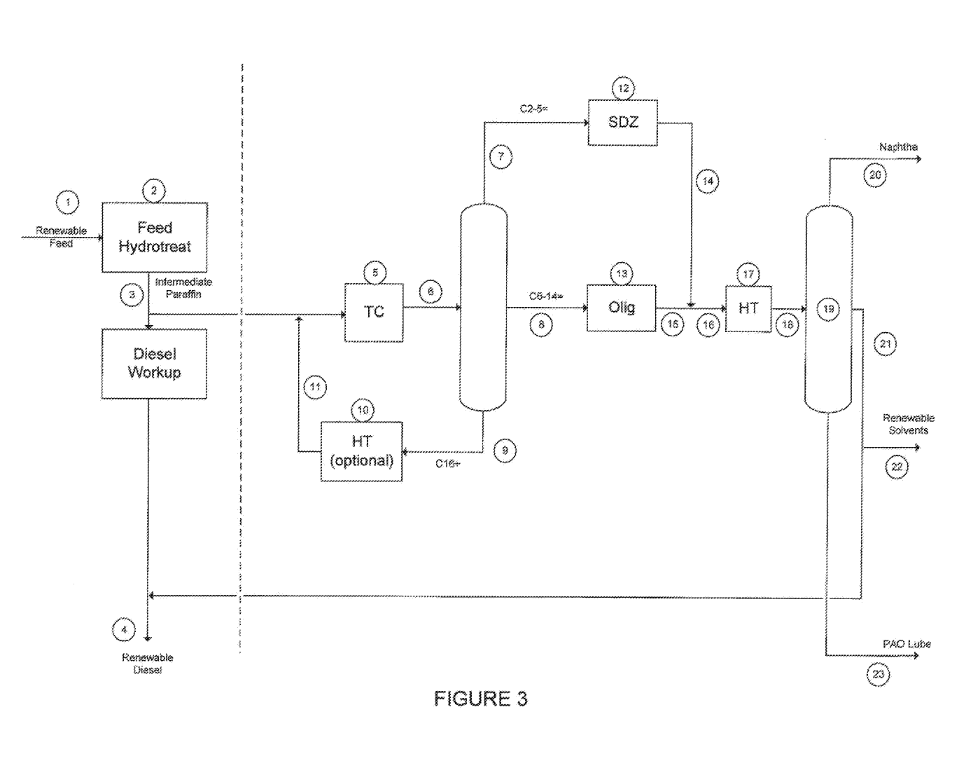

FIG. 3 is another preferred embodiment of the present invention. FIG. 3 depicts a process that consists of an existing renewable diesel facility with a new poly-alpha-olefin (PAO) production facility that uses an intermediate paraffin product from the renewable diesel facility as a feedstock. The same configuration could be a new stand-alone facility that produces both renewable diesel and PAO products. Renewable feed (1) is introduced to a feed hydrotreating unit (2) where triglycerides and/or free fatty acids are hydrotreated to produce products comprising C14 to C20 paraffins and light gases, including propane, water, CO and CO2. The paraffin products are separated from the light gases and split (in a limited embodiment the split may include 100% of the paraffin feed going to the PAO process) for use as renewable diesel feed or as feed to the PAO process where they are combined with recycled paraffin product (11) and fed to a thermal cracker (4).

Recycled paraffin product (11) is optionally hydrotreated to saturate any olefins in a hydrotreater (10).

Thermal cracked product (5) including light olefins and un-cracked paraffins is transferred to a distillation column (6) where light and intermediate olefins are separated from un-reacted paraffins. Light olefins (C2-C5) are removed and transferred via line (7) to a reactor (12) which contains a surface deactivated zeolite catalyst. The light olefins are oligomerized in the reactor (12), resulting in slightly branched C6 to C50+ products containing internal olefins. Intermediate range linear alpha olefins approximately (C6-C14) separated in the distillation column (6) are removed and transferred via line (8) to the main oligomerization reactor (13) where they are oligomerized with an oligomerization catalyst, preferably BF3 and a co-catalyst, to produce PAO products. These PAO products (15) are blended with PAO products (14) from reactor (12) and transferred via line (16) to a hydrotreater (17) where the olefins are saturated. The saturated product is transferred via line (18) to distillation column (19) where they are separated into naphtha (20), distillate (21) and PAO Lube Basestock (23) fractions. The distillate product (21) can be sold as one or more solvent products (22) which may require further distillation into narrow cuts or as a renewable diesel product (24).

The present invention makes it possible to convert paraffin feeds, and in a very limited embodiment, ethanol into a range of olefins from C2 to C30 or greater. Through multiple steps, the olefins can be modified to provide a majority of the olefins in a desired range [C7-C17], for example, for use as feed to oligomerization to Group IV basestocks. While multiple steps are identified to yield a majority of alpha olefins in the desired range, it is not outside the scope of the invention to leave out one or more of the steps.

Whereas, the present invention has been described in relation to the drawings attached hereto, it should be understood that other and further modifications, apart from those shown or suggested herein, may be made within the spirit and scope of this invention.

What is claimed is:

1. A process to produce poly-alpha-olefin (PAO) basestocks from a paraffin hydrocarbon feed comprising the steps of:
a. hydrotreating a renewable feed comprising triglycerides and/or free fatty acids to produce a paraffin intermediate product and light gases including water, carbon monoxide, carbon dioxide, and propane;
b. separating the paraffin intermediate product from the light gases;
c. thermal cracking the paraffin intermediate product to produce alpha olefins in the C2 to C16 range, where the alpha olefins comprise light olefins in the approximately C2 to C6 range and intermediate olefins in the approximately C6 to C16 range;
d. oligomerizing the light olefins (approximately C2-C6) from step (c) into higher molecular weight products, including slightly branched internal olefins, using a surface deactivated zeolite catalyst;
e. oligomerizing the intermediate olefins (approximately C6-C16) from step (c) with an oligomerization catalyst; and
f. hydrotreating and distilling products from steps (d) and (e) to produce finished products, including naphtha, distillates, solvents, and lube basestocks.

2. The process as set forth in claim 1 further comprising, after step (c) and prior to step (d), oligomerizing C2 olefins into higher carbon number linear alpha olefins for use in step (e).

3. The process as set forth in claim 1 wherein the surface deactivated zeolite comprises ZSM 5, ZSM 11, ZSM 23, or ZSM 48.

4. The process as set forth in claim 1 where step (c) further comprises recycling paraffin products to a thermal cracker.

5. The process as set forth in claim 4 further comprising hydrotreating recycled paraffin products.

6. The process as set forth in claim 1 further comprising finishing the distillates as a renewable diesel product or distilling the distillates into narrow cuts to produce renewable solvents products.

7. The process as set forth in claim 1 where step (f) further comprises hydrotreating and distilling the oligomerized products from the surface deactivated zeolite together with or separate from products produced by oligomerization of the higher molecular weight olefins.

8. The process as set forth in claim 1 where step (d) further comprises modifying by hydroformylation and dehydration the intermediate olefins (C6-C16) produced by the surface deactivated zeolite for use in step (e).

9. The process of claim 1 where the oligomerization catalyst is BF3 and a co-catalyst.
